(19) United States
(12) Patent Application Publication
(10) Pub. No.: US 2015/0250702 A1
(43) Pub. Date: Sep. 10, 2015

(54) EMOLLIENT-CONTAINING AQUEOUS GELS

(71) Applicant: ROHM AND HAAS COMPANY, Philadelphia, PA (US)

(72) Inventor: Curtis Schwartz, Ambler, PA (US)

(21) Appl. No.: 14/434,928

(22) PCT Filed: Oct. 2, 2013
(86) PCT No.: PCT/US13/63022

§ 371 Date: Apr. 10, 2015

Related U.S. Application Data
(60) Provisional application No. 61/714,367, filed on Oct. 16, 2012.

(51) Int. Cl. A61K 8/73; A61K 8/49; A61K 8/24; A61K 8/04; A61Q 1/06; A61Q 17/04; A61Q 1/02; A61Q 1/10; A61Q 1/04; A61K 8/06; A61Q 19/00

(52) U.S. Cl. CPC A61K 8/737; A61K 8/062; A61K 8/064; A61K 8/731; A61K 8/498; A61K 8/24; A61K 8/042; A61K 8/733; A61K 8/73; A61Q 19/00; A61Q 17/04; A61Q 1/02; A61Q 1/10; A61Q 1/04; A61Q 1/06; A61K 2800/33; A61K 2800/594; A61K 2800/592

(57) ABSTRACT

Described are personal care compositions, comprising a emollient-containing gel formed from an aqueous gel composition comprising: a gel forming blend, comprising: a) methylcellulose, hydroxypropyl methylcellulose, carboxymethylcellulose, or mixtures thereof, and b) xanthum gum, gellan, gelatin, pectin, carrageenan, alginic acid, alginates, curdlan, guar gum, fenugreek gum, locust bean gum, konjac gum, agarose, or mixtures thereof, in a weight ratio of from 1:8 to 8:1; and from 1 to 40 wt. % of a emollient; provided that emollient-containing gel displays shear thinning behavior.

### CROSS-REFERENCE TO RELATED APPLICATION(S)

This application claims priority to and is a 371 U.S.C. §371 national phase application of International Application No. PCT/US13/063022, filed on Oct. 2, 2013, which claims the priority benefit of U.S. Provisional Application No. 61/714,367 filed on Oct. 16, 2012, each of which is incorporated herein by reference in its entirety

### FIELD

The present invention relates to a personal care compositions, and methods for moisturizing with emollients.

### BACKGROUND

In the personal care field, there is a need for materials which encapsulate or entrain emollients and then, when applied to a person, release the emollients, providing a moisturizing sensation. However, there are a number of considerations which demand attention from the industry, including a need for high active content, suitable resistance to heat, appropriate shear thinning behavior and aesthetics, desirable water retention, and environmental compatibility.

Accordingly, what is needed are new emollient systems which address the above-described needs.

### DETAILED DESCRIPTION

In one embodiment, the present invention provides personal care compositions, comprising a emollient-containing gel formed from an aqueous gel composition comprising: a gel forming blend, comprising: a) methylcellulose, hydroxypropyl methylcellulose, carboxymethylcellulose, or mixtures thereof, and b) xanthum gum, gellan, gelatin, pectin, carrageenan, alginic acid, alginates, curdlan, guar gum, fenugreek gum, locust bean gum, konjac gum, agarose, or mixtures thereof, in a weight ratio of from 1:8 to 8:1; and from 1 to 40 wt. % of a emollient; provided that emollient-containing gel displays shear thinning behavior.

“Personal care” relates to compositions to be topically applied to a person, but not ingested. Preferably, the personal care composition is a skin care product (e.g., facial cream, moisturizers, leave on and rinse off lotions, sunscreens, foundation, mascara, eye-liner, lipstick, and the like). In one embodiment, the personal care composition is substantially free of surfactants.

“Aqueous gel composition” refers to the fact that the major component is water. In one embodiment, at least 20% by weight of the aqueous gel composition (“wt. %”) is water, preferably at least 40 wt. %, more preferably at least 50 wt. %, more preferably at least 60 wt. %, more preferably at least 70 wt. %, more preferably at least 80 wt. %, up to 90 wt. %.

It is understood that the aqueous gel composition sets, cures, cross-links, or otherwise gels to form the emollient-containing gel. Accordingly, the emollient-containing gel may vary in hardness, but in any case, the gel must display shear thinning behavior. “Shear thinning behavior” means the viscosity of the gel decreases with increasing shear rate, such as when applying the product onto skin.

The gel forming blend comprises a part a) comprising methylcellulose, hydroxypropyl methylcellulose, carboxymethylcellulose, or mixtures thereof.

In one embodiment, the methylcellulose has an average degree of substitution DS_{methyl} of from 1.2 to 2.0, more preferably from 1.5 to 1.9 and most preferably from 1.7 to 1.9. Typically, viscosities of 2% by weight aqueous methylcellulose solutions at 20° C., range from 40 to 80,000 mP·s, preferably from 1,000 to 78,000 mP·s, and more preferably from 15,000 to 75,000 mP·s. Examples of commercially available methylcelluloses that are useful in the present invention include METHOCEL™ A, SGA, E, K, and G series; especially preferred is METHOCEL™ A40M (DS_{methyl}=1.8, 2% by weight viscosity=40,000 mPa·s,) available from The Dow Chemical Company, Midland, U.S.A.

In one embodiment, the hydroxypropyl methylcellulose has an average degree of substitution DS_{methyl} of from 1.2 to 2.0, more preferably of from 1.3 to 1.8, and most preferably from 1.3 to 1.5 and a molar degree of substitution MS_{hydroxypropyl} of from 0.1 to 0.25, more preferably of from 0.15 to 0.25, and most preferably of from 0.20 to 0.23. Typically, viscosities of 2% by weight aqueous hydroxypropyl methylcellulose solutions at 20° C., range from 15 to 250,000 mPa·s, preferably from 450 to 200,000 mPa·s, and more preferably from 4,000 to 180,000 mPa·s. Examples of commercially available hydroxypropyl methylcelluloses that are useful in the present invention include METHOCEL™ K100M (DS_{methyl}=1.4, MS_{hydroxypropyl}=0.21, 2% by weight viscosity=100,000 mPa·s), and more preferably METHOCEL™ K15M (DS_{methyl}=1.4, MS_{hydroxypropyl}=0.21, 2% by weight viscosity=15,000 mP·s), available from The Dow Chemical Company, Midland, U.S.A.

In one embodiment, the gel forming component is carboxy methylcellulose. In one embodiment, the carboxy methylcellulose has a molar degree of substitution MS_{carboxy} of from 0.5 to 1.2, more preferably of from 0.6 to 1.1, and most preferably of from 0.7 to 0.95. Typically, viscosities of 1% by weight aqueous carboxy methylcellulose solutions at 20° C., determined with a Brookfield viscometer, range from 20 to 50000 mPa·s, preferably from 500 to 2000 mPa·s, and more preferably from 2000 to 10000 mPa·s. Examples of commercially available carboxy methylcelluloses that are useful in the present invention include WALOCEL™ CRT 50000 PA (MS_{carboxy}=0.7, 1% by weight Brookfield viscosity=7000 mPa·s), and more preferably CLEAR+STABLE (C&S) 30M (MS_{carboxy}=0.9, 1% by weight Brookfield viscosity=2,700-4,900 mP·s), available from The Dow Chemical Company, Midland, U.S.A.

In some embodiments the composition is free of any cellulose derivative other than methylcellulose, hydroxypropyl methylcellulose, or carboxymethylcellulose. Further, in some embodiments, when one of methylcellulose, hydroxypropyl methylcellulose, or carboxymethylcellulose is selected, the composition is free of the other two cellulose derivatives.

The gel forming blend comprises a part b) comprising xanthum gum, gellan, gelatin, pectin, carrageenan, alginic acid, alginates, curdlan, guar gum, fenugreek gum, locust bean gum, konjac gum, agarose, or mixtures thereof.

In one embodiment, part b) is selected from non-ionic and non-acidic polysaccharide hydrocolloids.

In one embodiment, part b) is selected from ionic and acidic polysaccharide hydrocolloids. In a further embodiment of this embodiment, an optional salt is included to cross-link or precipitate with metal ions. “Salt” refers to at least one inorganic cation. Preferably, the salt is a divalent cation such as for example Ca²⁺, Mg²⁺, and/or Zn²⁺ cations. Examples of suitable gel-promoting salts include calcium phosphate, calcium hydrogen phosphate, and mixtures thereof. If calcium phosphate, calcium hydrogen phosphate or another hydrogen phosphate or hydrogen phosphate of low solubility is used, glucono delta-lactone (GDL) may be added which gradually reacts with the (hydrogen) phosphate to release the cation. A preferred amount of GDL is within the range of from 0.01 to 2 wt. %. The salt is preferably from 0.01 to 5 wt. % of the aqueous gel composition, more preferably from 0.05 to 3 wt. %, more preferably from 0.1 to 2 wt. %, more preferably from 0.1 to 0.5 wt. %

In one embodiment, the gel forming blend has a part a) to part b) weight ratio of from 1:8 to 8:1, preferably from 1:2 to 6:1, preferably 1:1 or alternatively, in some embodiments, 4:1.

In one embodiment, the gel forming blend is preferably present in the from 0.5 to 5 wt. %, more preferably from 1 to 4 wt. %, and most preferably from 1.5 to 3 wt. %.

Examples of emollients include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, synthetic esters and ethers, especially of fatty acids, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), ethers such as dicaprylyl ether, and mixtures thereof.

Oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, and the oil of cereal germs.

Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. In a preferred embodiment, the emollient is at least one of myristates, stearates, palmitates, cocoates, coco-caprylate/caprates, oleates, hexyldecanol, octyldodecanol, caprylic/capric triglycerides, or cocoglycerides, and their alkoxylates. The emollient is in an amount of from 1 to 60 wt. % of the aqueous gel composition, preferably from 10 to 40 wt. %, more preferably from 5 to 25 wt. %, and even more preferably from 9 to 22 wt. %.

Optional ingredients include those conventionally used in personal care emollient releasing compositions, including preferably cosmetically acceptable sunscreens, emulsifiers, rheology modifiers, colorants, dyes, pH adjustors, reducing agents, fragrances, anti-foaming agents, antiseptics, deodorants, antiperspirants, insect repellants, fillers, barrier materials, or preservatives. “Cosmetically acceptable” refers to ingredients typically used in personal care compositions, and is intended to underscore that materials that are toxic when present in the amounts typically found in personal care compositions are not contemplated as part of the present invention.

The composition according to the present invention may be prepared by several methods known in the art. One exemplary route is to first prepare separate colloidal solutions of each of the cellulose ether and the part b) component and then combine those colloidal solutions and add further optional ingredients. Another exemplary route is to first dry mix the cellulose ether and the part b) component and then prepare a colloidal solution of the mixture and add further optional ingredients.

In one embodiment, the composition is emulsified before gelling. Typically, the oil-containing compositions of the present invention form stable oil-in-water emulsions. In one embodiment, emulsifying salts may be added in addition to the gel-promoting salts described above. Examples of suitable emulsifying salts include trisodium phosphate, disodium hydrogen phosphate, sodium dihydrogen phosphate, sodium citrate, and alkaline sodium aluminum phosphate. Further exemplary ingredients that may be contained in the present composition include processing agents, such as emulsifiers different from emulsifying salts.

The compositions of the present invention have comparable physical properties to traditional cream, lotion, or ointment products, including appearance, texture, rheology, and spreadability. During their preparation no or only minimal undesired foaming is observed at high shear blending operations. Thus, the final product is largely bubble-free. The present composition can be manufactured with a wide range of texture rheology and elasticity. In some embodiments, compositions of the present invention form a shear thinning gel.

### EXAMPLES

### Example 1

Compositions of the present invention are described in Tables 1A and 1B, having the components listed in wt %.

TABLE 1A

| | Batch | Batch | Batch | Batch | Batch | Batch |
|---|---|---|---|---|---|---|
| | 1 | 2 | 3 | 4 | 5 | 6 |
| METHOCEL A4M | 1 | 0.8 | 0.8 | 0.8 | 1 | — |
| Methylcellulose | | | | | | |
| Carboxy | — | — | — | — | — | 1 |
| methylcellulose | | | | | | |
| Alginate | 1 | 0.2 | 0.2 | 0.2 | 1 | 1 |
| Konjac gum | — | — | 0.2 | — | — | — |
| Curdlan gum | — | — | — | 0.2 | — | — |
| Carrageenan | — | — | — | — | 0.5 | — |
| Shea butter oil | 20 | 20 | 20 | 20 | 20 | 20 |
| CaHPO₄ | 0.5 | 0.18 | 0.18 | 0.18 | 0.5 | 0.5 |
| Glucono delta- | 0.5 | 0.4 | 0.4 | 0.4 | 0.5 | 0.5 |
| lactone | | | | | | |
| Water | 77 | 78.42 | 78.22 | 78.22 | 76.5 | 77 |

TABLE 1B

| | Batch 7 | Batch 8 | Batch 9 | Batch 10 | Batch 11 |
|---|---|---|---|---|---|
| METHOCEL A4M | 0.5 | 0.5 | 0.5 | 1 | — |
| Methylcellulose | | | | | |
| Carboxy | — | — | — | — | 1 |
| methylcellulose | | | | | |
| Alginate | 0.5 | 0.5 | 0.5 | — | 1 |
| Carrageenan | — | — | — | 0.5 | — |
| Jojoba oil | 10 | 10 | 10 | 20 | 5 |
| CaHPO₄ | 0.3 | 0.3 | 0.3 | 0.5 | 0.5 |
| Glucono delta- | 0.4 | 0.4 | 0.4 | 0.5 | 0.5 |
| lactone | | | | | |
| Water | 78.3 | 78.3 | 78.3 | 76.5 | 92 |

First, a solution of methylcellulose in water is prepared by weighing methylcellulose into a beaker and carefully pouring in distilled water at a temperature of 95° C. The mixture is stirred for 5 min at 1100 rpm, then cooled first in tap water and then in ice water. Afterwards, the solution is stirred for 10 to 15 min at 1100 rpm.

An alginate or alginate/carrageenan solution is prepared by adding salt under stirring into water and stiffing is continued at 1500 rpm for 2 min at 20° C. The solution is heated to 90° C. and stirred at 2000 rpm for 5 min. Then, the solution is cooled in ice water under stirring at 2000 rpm for 10 min

Corresponding amounts of the methylcellulose solution including the emollient and alginate or alginate/carrageenan solution to achieve the concentrations as specified in Table 1 are combined and mixed under stiffing at 2000 rpm for 10 min A 20 mmol/l solution of GDL in water is added and stirring is continued at 2000 rpm for 5 min

All samples were well emulsified, which produced smooth compositions with no oily surface, and all gelled at room temperature within 3 hours.

The samples were put into water, and no emollient oil is observed in the water, demonstrating that the emollient is stably encapsulated by the compositions. Furthermore, the gel components can be optimized to obtain spreadability and shear thinning

### Example 2 (Comparative)

Compositions outside of the present invention are described in Table 2, having the components listed in wt %.

TABLE 2

| | Comparative | Comparative | Comparative |
|---|---|---|---|
| | Batch A | Batch C | Batch D |
| METHOCEL A4M | 0.8 | 0.5 | 0.5 |
| Methylcellulose | | | |
| Konjac gum | 0.2 | — | — |
| Curdlan | — | 0.2 | — |
| Guar gum | — | — | 0.2 |
| CaCl₂ | — | 0.4 | — |
| CaHPO₄ | — | — | — |
| Glucono delta- | — | — | — |
| lactone | | | |
| Water | 99 | 98.9 | 99.3 |

Comparative Batches A, B, and C used methylcellulose in combination with konjac gum, curdlan, or guar gum, but displayed phase separation even in absence of emollient.

1. A personal care composition, comprising a emollient-containing gel formed from an aqueous gel composition comprising:
a gel forming blend, comprising:
a) methylcellulose, hydroxypropyl methylcellulose, carboxymethylcellulose, or mixtures thereof, and
b) xanthum gum, gellan, gelatin, pectin, carrageenan, alginic acid, alginates, curdlan, guar gum, fenugreek gum, locust bean gum, konjac gum, agarose, or mixtures thereof,
in a weight ratio of from 1:8 to 8:1; and
from 1 to 40 wt. % of a emollient;
provided that emollient-containing gel displays shear thinning behavior.

2. The personal care composition of claim 1, wherein the gel forming blend is present in a range from 1.5 to 3 wt. %.

3. The personal care composition of claim 1, the weight ratio of a) to b) is 1:1.

4. The personal care composition of claim 4, further comprising a salt.

5. The personal care composition of claim 4, further comprising glucono delta-lactone, wherein the salt is selected from calcium phosphate, calcium hydrogen phosphate, and mixtures thereof.

6. The personal care composition of claim 1, comprising greater than 70 wt. % water.

7. The personal care composition of claim 1, comprising 20 wt. % emollient.

8. The personal care composition of claim 1, the personal care composition is a oil in water emulsion.

9. The personal care composition of claim 1, the personal care composition is a water. in oil emulsion.
